(19) United States
(12) Patent Application Publication
(10) Pub. No.: US 2017/0312576 A1
(43) Pub. Date: Nov. 2, 2017

(54) Wearable Physiological Sensor System for Training and Therapeutic Purposes

(71) Applicants: Senthil Natarajan, Houston, TX (US); John Frullo, Houston, TX (US)

(72) Inventors: Senthil Natarajan, Houston, TX (US); John Frullo, Houston, TX (US)

(21) Appl. No.: 15/477,087

(22) Filed: Apr. 1, 2017

Related U.S. Application Data
(60) Provisional application No. 62/317,505, filed on Apr. 2, 2016.

(51) Int. Cl. A63B 24/00; A61B 5/00; A61B 5/11; A61B 5/04; A63B 71/06; A61B 5/0488

(52) U.S. Cl. CPC A63B 24/0062; A63B 71/0619; A61B 5/6804; A61B 5/0488; A61B 5/04012; A61B 5/486; A61B 5/11; A61B 2503/10; A61B 2562/0219

(57) ABSTRACT

Wearable systems and methods to comprehensively analyze physical activity of a user for training and/or therapeutic purposes, by analyzing multiple channels of data about both muscle activity, using non-invasive surface electromyography (sEMG), and associated motion from that muscle activity, using inertial measurement units (IMU), are disclosed.

This application claims priority under 35 USC 119(a)(1) of Provisional Application 62/317,505 filed Apr. 2, 2016, the contents of which are herein incorporated by reference.

### FIELD OF THE INVENTION

The present disclosure relates to a wearable system and methods to comprehensively analyze physical activity of a user for training and/or therapeutic purposes, by analyzing multiple channels of data about both muscle activity, using non-invasive surface electromyography (sEMG), and associated motion from that muscle activity, using inertial measurement units (IMU).

### BACKGROUND OF THE INVENTION

Most applications of electromyography in training and therapeutic settings are restricted to hospital and clinical settings instead of real-time, portable form factors. Training devices that solely incorporate motion tracking and analysis suffer from a lack of consistent, reliable data and only serve as approximations of the true nature of actual physical stress in a body. Devices that solely incorporate EMG may suffer failure in the presence of externalities such as sweat and dust, and are not utilized currently in therapeutic settings. Although a combination of EMG and IMU sensors has been previously proposed, those applications were primarily for detecting gestures and subsequently using those gestures to control another device or component.

US Patent Applications 20150169074, 20140240103, and 20130317648A1 all disclose inventions that combine electromyography sensors with inertial motion units. US Patent Applications 20150169074 and 20140240103 from Thalmic Labs primarily focus on a band of sEMG electrodes used for gesture recognition and subsequent device control, as well as the computational algorithms behind the gesture control. US Patent Application 20130317648A1 discloses a sleeve with an embedded array of sensors which are used for gesture recognition and robotics control applications. However, these foregoing disclosed inventions focus on gesture identification and classification.

US Patent Applications 20150105882, 20140156218, 20060025229 and U.S. Pat. No. 7,492,367, all disclose systems and methods for capturing and analyzing IMU data, however they are specific to motion tracking and do not incorporate muscle analysis. U.S. Pat. No. 9,498,128 also discloses a wearable system for performance monitoring focused on EMG analysis, however, while it makes allowances for the incorporation of various sensors into potential embodiments, IMU data analysis or the construction of such a system incorporating an IMU is not detailed.

U.S. Pat. No. 6,643,541 discloses a flexible wireless patch EMG sensor and provides a means of over-the-air communication between a plurality of sensors and a receiving system. However, while the patent mentions a Parkinson's test, no description of specific data analysis is disclosed and the application focuses more on the construction of such a sensor, as well as the communications interface rather than the use of an integrated biometric monitoring device. US Patent application 20120071732 discloses algorithms and techniques for sEMG data analysis, essential for any sort of progressive muscle fatigue analysis. However, the application is primarily software focused rather than hardware focused and is not about the construction of sensory systems which would utilize such analysis.

US Patent applications 20140259267 and 20030212319 disclose methods of incorporating biometric monitoring systems into apparel and health-monitoring garments. However, the disclosed inventions are focused primarily on the housing of sensors in the garments and the construction of such garments rather than the sensors or the analysis of the data provided by the sensors. U.S. Pat. No. 7,793,361 and US Patent application 20140039804A1 disclose systems for wearable biometric monitoring systems, such as wrist wearables which have become popular over the last few years by companies such as Nike and Fitbit. However, the disclosed inventions are focused primarily on the concept of housing biometric sensors in a wrist garment or similar apparel, rather than detailed methods for creation of the specific sensors or their use for actual direct measurement of muscle activity.

US Patent Applications 20100185398 and 20060136173 disclose systems for monitoring athletic activity, including wearable sensors, for analyzing athletic performance. However, while the disclosed inventions provide a generalized view of how such systems would need to be integrated in a real-time application and discuss the interaction between the user, sensors, communications systems, computation systems, and end-display devices, the patent applications do not detail the data analysis or the construction of such a sensory system. They do not provide details on the creation of such sensors, focusing rather on how those sensors would be incorporated in a system.

However, it is still clear that there is an unmet need for a wearable, real-time performance training and therapeutic device which provides analysis of muscle activity and its associated motion tracking using appropriate sensors.

### SUMMARY OF THE INVENTION

The present disclosure provides sensory systems and methods that may be used for monitoring the risk of arm injury for baseball pitchers, or other risk of injury for similar physical motions by athletes, like for example, but not limited to, tennis, soccer, or football. One embodiment provides a device that integrates both EMG and IMU sensors into a wearable form factor for biometric health monitoring and performance tracking, especially for use in an athletic training or therapeutic applications. This device focuses on health and biometric tracking applications, and as such extracts and analyzes an entirely different type of physiological data from the biometric sensors and monitors biometric risk factors for injury from physical activity

One baseball embodiment is an arm sleeve that, for example, monitors every pitch by collecting data from sensors on both muscle activity and its associated motion activity, then appropriately filtering, amplifying, and pre-processing that sensor data. The pre-processed sensor data may then be additionally processed and analyzed for various biological/physiological markers including, but not limited to: muscle fatigue, elbow height, torque about the elbow, and reconstructing the raw motion path of the arm to analyze throwing technique. The resulting analysis may then be communicated to a mobile device via a wireless protocol, such as Bluetooth, for review of the analyzed activity for training and/or therapeutic purposes, and/or for prevention of injury.

These and other features and advantages of the present disclosure will become apparent to those skilled in the art from the following detailed disclosure, taken together with the accompanying drawings.

### BRIEF DESCRIPTION OF THE DRAWINGS

The accompanying drawings, which are incorporated herein and form part of the disclosure, illustrate various embodiments of the present disclosure, and together with the description, further serve to explain the principles of the disclosure and to enable a person skilled in the art to make and use the embodiments disclosed herein. Certain embodiments of the disclosure will be described with reference to the accompanying drawings. However, the accompanying drawings illustrate only certain aspects of the disclosure by way of example and are not meant to limit the scope of the claims. In the drawings, like reference numbers indicate identical or functionally similar elements.

FIGS. 1A, B depict a simplified, representative sleeve for an exemplary embodiment.

FIG. 2 depicts a representative, simplified diagram for sensor locations in accordance with exemplary embodiments.

FIG. 3 depicts a system in accordance with exemplary embodiments.

FIGS. 4A, B depict raw sensor signals and partially processed data from those signals in accordance with exemplary embodiments.

FIG. 5 depicts a method for use in accordance with exemplary embodiments.

FIG. 6 depicts a baseball field dimensions for use in accordance with exemplary embodiments.

FIG. 7 depicts an exemplary system embodiment.

FIGS. 8A, B, C depict a method for exemplary embodiments.

FIG. 9 depicts a block diagram of components for an exemplary embodiment.

FIG. 10 depicts a block diagram for a portion of an exemplary embodiment.

FIG. 11 depicts a block diagram for a portion of an exemplary embodiment.

FIG. 12 depicts a block diagram for a portion of an exemplary embodiment.

FIG. 13 depicts a block diagram for a portion of an exemplary embodiment.

### DETAILED DESCRIPTION

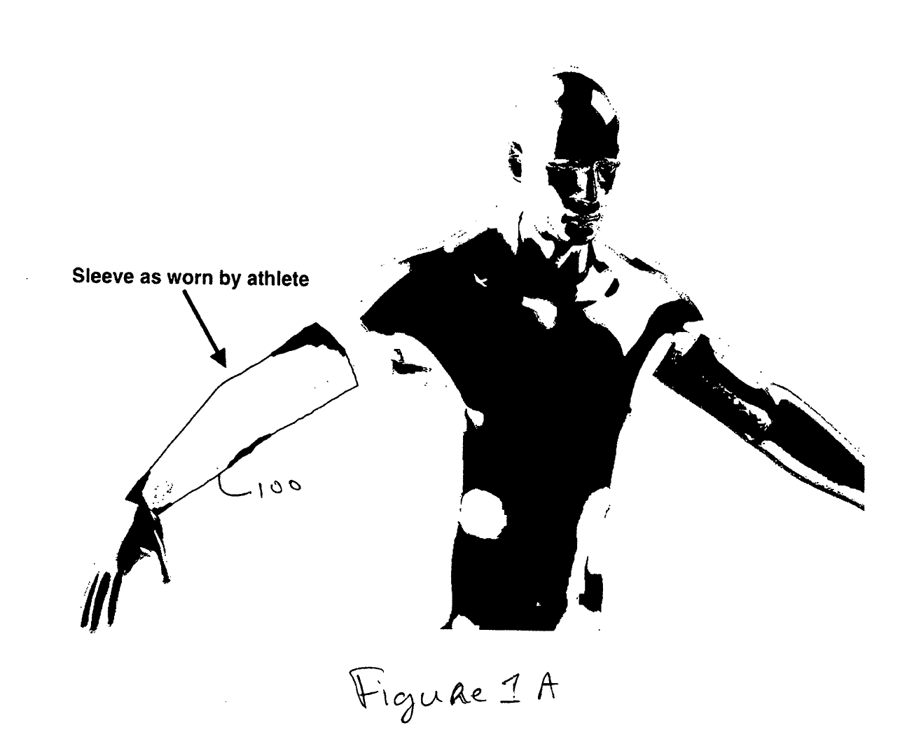

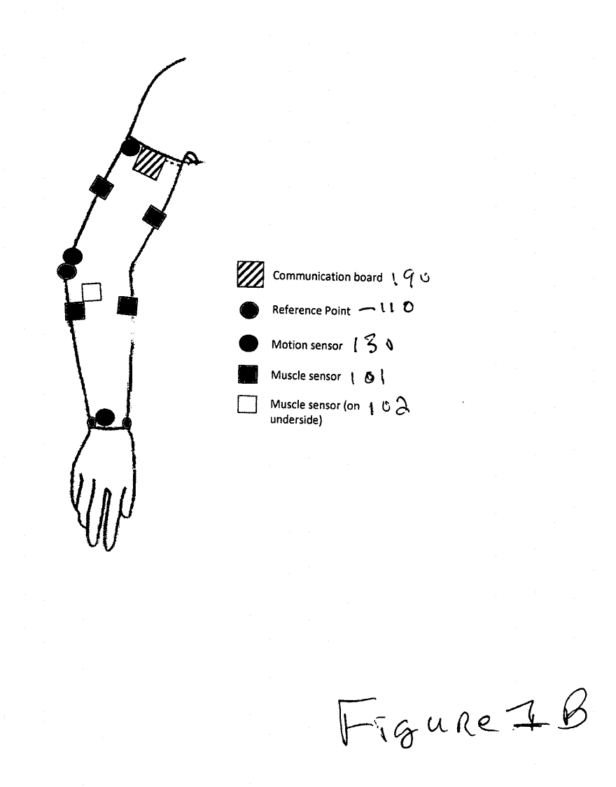

Referring now to FIGS. 1A, B, there may be seen one presently preferred embodiment of the disclosed sensory system that is an arm sleeve 100 for monitoring the risk of arm injury for baseball pitchers. This embodiment, for every pitch, collects data from the sensors on both muscle activity 101, 102 and its associated motion activity 130. As more fully described later herein, that data may then be filtered, amplified, and pre-processed. The pre-processed sensor data may then be additionally processed to generate various biological/physiological markers including, but not limited to: muscle fatigue, elbow height, torque about the elbow, and reconstructing the raw motion path of the arm to analyze throwing technique. The data may then be communicated to a mobile device via a wireless protocol, such as Bluetooth, or any available and operative wireless telephony system. Using the sensor data, this embodiment preferably processes the data and monitors various biological/physiological markers for potential injury, but before an injury actually occurs; these markers include, but are not limited to: muscle fatigue, elbow height, torque about the elbow, and reconstructing the raw motion path of the arm to analyze throwing technique. Optionally, sensor data may be transmitted to a central cloud-based processing and data storage facility for additional processing and computation, where the processed data is translated to actionable feedback and transmitted to a mobile device for an end user to monitor. This embodiment is intended to be used in both practice sessions and in-game, providing real time analysis that continually adds to and uses a user's prior historical body of data. While the pitcher is wearing the device, a coach or trainer will be able to monitor the resultant feedback to determine muscle fatigue and for appropriate pitching motion technique.

Continuing to refer to FIG. 1A, this embodiment of the disclosure is a garment in the form of an elastic sleeve 100 to be worn on either arm. As depicted in FIG. 1B, the sleeve 100 is configured to hold at least one sensor, in a permanent or releasable fashion. Each sensor may acquire data relevant to one or more areas of useful biometric information, including, but not limited to, muscle activity and/or motion associated with that muscle activity. The sleeve may have an appropriate on/off switch located thereon.

Currently there are eight sensors on a preferred embodiment of the sleeve: five for muscle activity 101, and three for motion 130; however more or fewer such sensors may be so employed. The sensors may be separate (i.e. a separate muscle sensor and a separate motion sensor) but may also be combined into one “sensor unit”. Further, additional sensor units may be included as part of a device, especially as the garment containing sensors scales up from a sleeve to a shirt or leggings, such as for example, but not limited to a heart rate monitor.

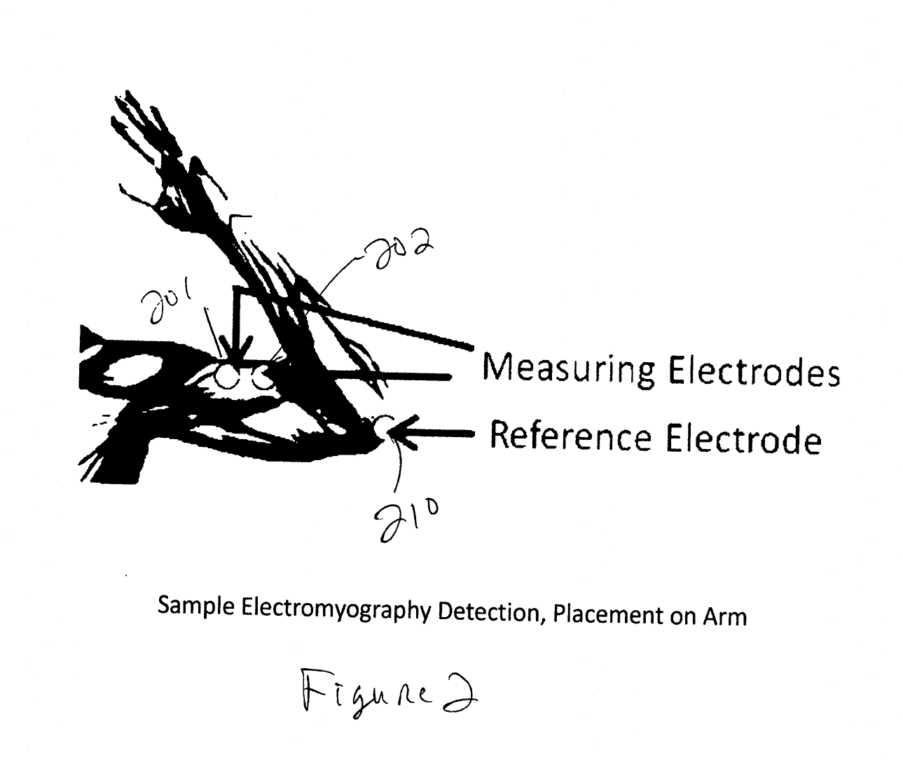

Each muscle activity sensor uses metallic electrodes that contact the skin surface of a user to collect the nerve signals causing the muscle activity, typically using a surface electromyography (sEMG) sensor. FIG. 2 depicts a simplified but representative placement for multiple sMEG sensors on an arm for an embodiment of the disclosure like that in FIGS. 1. One embodiment of each muscle sensor 201, 202 will consist of two rectangular silver/silver chloride electrodes, each measuring 10 mm by 1 mm. These will be spaced 10 mm apart in a parallel fashion (similar to the Delsys electrode design: http://www.delsys.com/products/desktop-emg/surface-emg-sensors/) and will be permanently attached to a muscle activity sensor unit (described more fully later herein). Other embodiments of the muscle activity sensor may include electrodes connected permanently to a sensor unit (circuit board) or attached via some sort of removable method (via, for example, snap on clips, etc.). Additionally, the electrodes could be any variety of metal or a combination thereof, including, but not limited to, copper, silver, gold, and/or titanium. A reference electrode 210 is also depicted.

As described more fully later herein, the signal detected by the sensor electrodes is then conditioned by additional components of its sensor unit (typically signal conditioning and processing may be accomplished by both analog and digital circuitry). Specifically, the electrodes capture the raw nerve signal causing muscle activity and then pass this relatively small-scale signal on to a sensor unit for amplification and filtering before conversion from analog to digital. After conversion, additional conditioning (filtering/processing), may occur within a microprocessor located in the sensor unit before the signal is passed out of the sensor unit to other components for collection, storage, and/or further processing, and then for data transmission.

As depicted in FIG. 2, in order to properly reference the sEMG signals measured by the muscle sensor units 201, 202, conductive material 210 will also be placed in contact with the skin at the elbow and/or wrist bones, as a reference electrode. These sensor contact points serve as a reference to properly orient and calibrate the sleeve for repeatable, accurate measurements. The user of the sleeve embodiment will be able to align their wrist and elbow bones with the sensor contact points for appropriate positioning of the muscle activity sensors. Additionally, orientation and alignment lines or designs may be placed along the sleeve exterior so that, when aligned a certain way, it ensures proper positioning of the sensors. Finally, an optional software component demonstrating use of the sleeve may include a visualization of the arm and details of proper placement of the sleeve to provide for correct positioning of the sleeve on the arm.

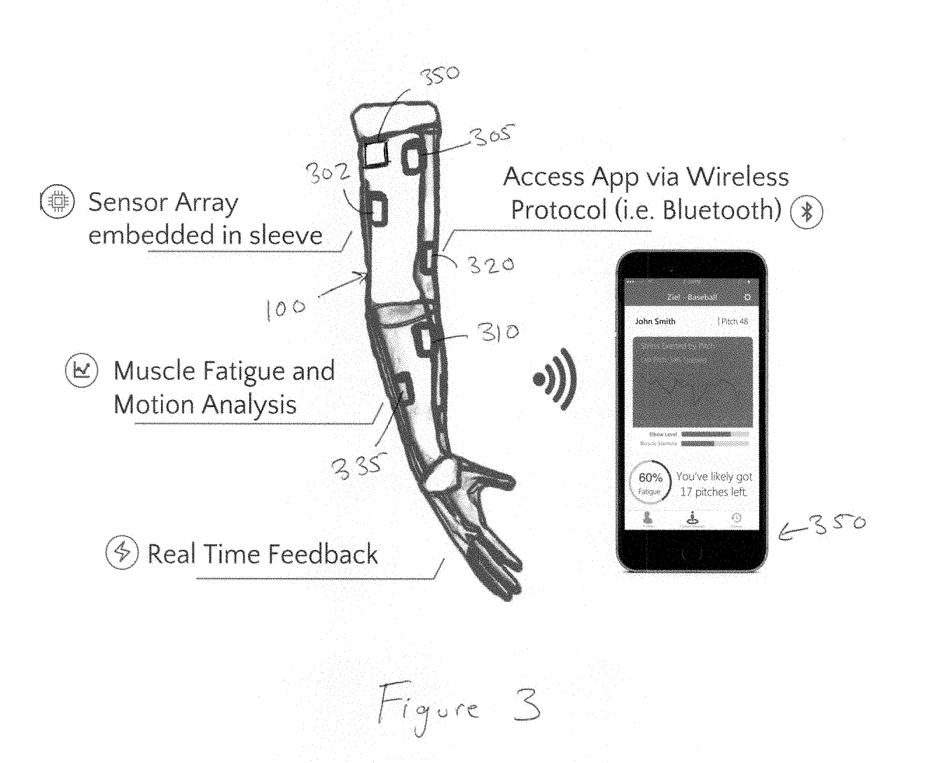

As depicted in FIG. 3, each motion sensor 305, 310 uses elements (either comprised of one or more Inertial Measurement Unit(s) (IMU) components or individual components [accelerometer, gyroscope, magnetometer]) to determine motion characteristics of that particular sensor location on a user appendage (position and orientation) over time. The motion information signals produced by these motion sensor components are appropriately filtered and amplified in a sensor unit, and then passed to a local microprocessor in the sensor unit, which may be the same, or a different, processor mentioned above for muscle signal processing, for further conditioning and gathering before it too is passed out of the sensor unit to be collected, stored and/or further processed, and then the data may be transmitted to other facilities or components for further analysis, storage, or use.

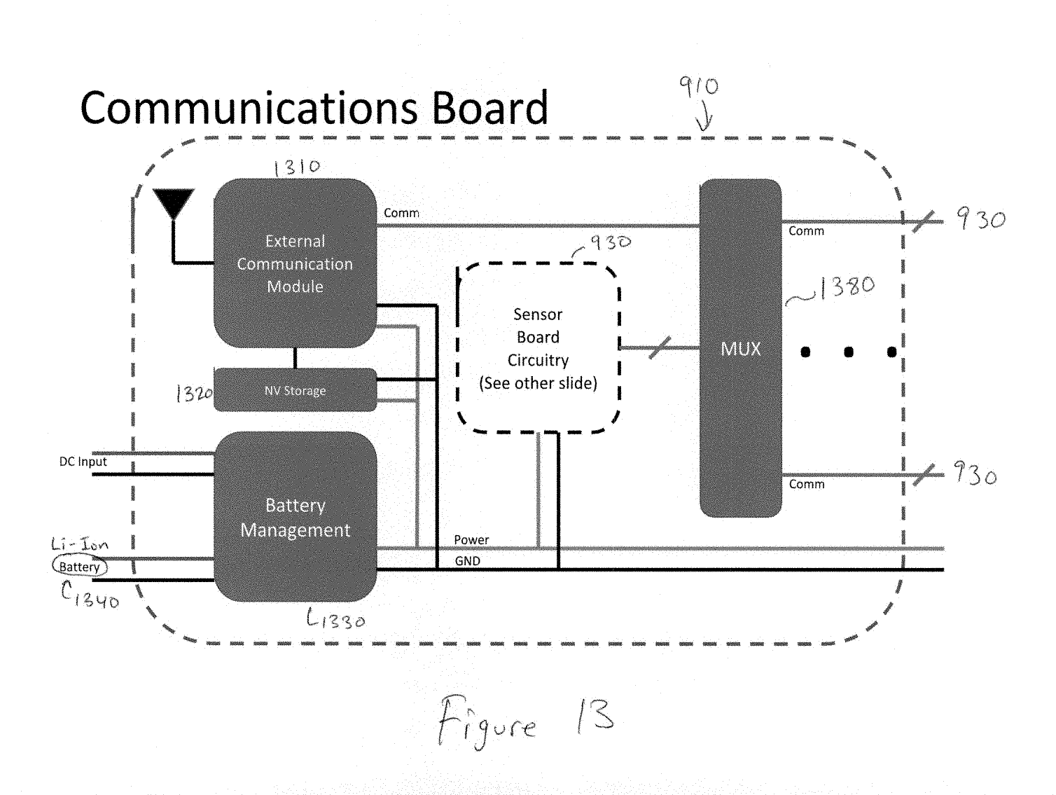

As depicted in FIG. 3, a central communications unit 350 may be positioned on the sleeve garment 100 and houses a component that acquires all the sensor information in a time-synchronized manner. The gathered information is then transmitted out from the communications unit via Bluetooth (through a Bluetooth Low Energy [BLE] module contained in unit 350). Although the central communications unit may be on the sleeve 100 as depicted, it may also optionally be located elsewhere on the user in a convenient and non-obstructive location while still being operatively connected to any sensor units. The garment 100 is also equipped with a miniaturized, rechargeable lithium ion battery with a battery management component responsible for monitoring battery life. Furthermore, the garment also contains a component for local non-volatile storage of collected information (as described herein later with reference to FIG. 13). In the event that the garment is not able to securely connect with an external device, the non-volatile storage component of the system will store the collected data until a connection is established and the data may be transmitted. Transmission may be made using any of the available wireless telephony networks and then through other internet equipment available to those networks on to cloud based processing and/or storage components.

All the system components/units may be contained within the sleeve 100 in either a permanent or removable manner, with each sensor unit being contained on either a separated flexible circuit board or all units connected together on one flexible circuit board that spans the length of the garment. Other embodiments may locate the battery, battery management unit and communications unit in a separate housing or garment for placement in other locations on a user while remaining operatively connected with the sensor and other units or components using removable wire cables, or other suitable interconnectors. The sleeve 100 will also ideally be made of such material that it is compatible with use on either arm without any necessary hardware reconfiguration. This also enables any supporting software to determine which arm the sleeve is worn on, as an added benefit. However, the sleeve may also be designed such that it is configured for a given arm, especially if additional sensors are added for higher performance embodiments.

As depicted in FIG. 3, one embodiment of the data transmission aspect of the system includes the garment 100 transferring data over Bluetooth to a user mobile device 350. Optionally, this mobile device may then stream the data to a cloud-based server for additional processing and storage. Any processed information is then passed back to the mobile device to be displayed for use. This method also allows local storage of the information on the mobile device, if connection to the cloud server is unavailable. The data may be transmitted once connection is restored for later processing. Additional embodiments involve the garment initially bypassing the mobile device and streaming directly to the cloud server for processing and storage and then later transmission to the mobile device for use.

In an alternate embodiment, the data transmission would take place via a wireless protocol such as Wi-Fi or Bluetooth between the sensory sleeve and a user's mobile device, and all processing and computation would occur locally, handled by the processor unit in the sensory sleeve and the mobile device. Finally, the data would then be sent to a remote cloud server, but only for storage and database purposes, and for later review, and, optionally, further analysis.

The garment 100 is configured to collect useful information from the wearer during physical activity in real-time and/or following the event via post-processing of the stored information. All collected data is user-based, such that the user can login from any device (mobile device/PC) in real-time or post-activity to analyze the measurements, as long as that device has been granted access to the user's data (security is more fully discussed herein below).

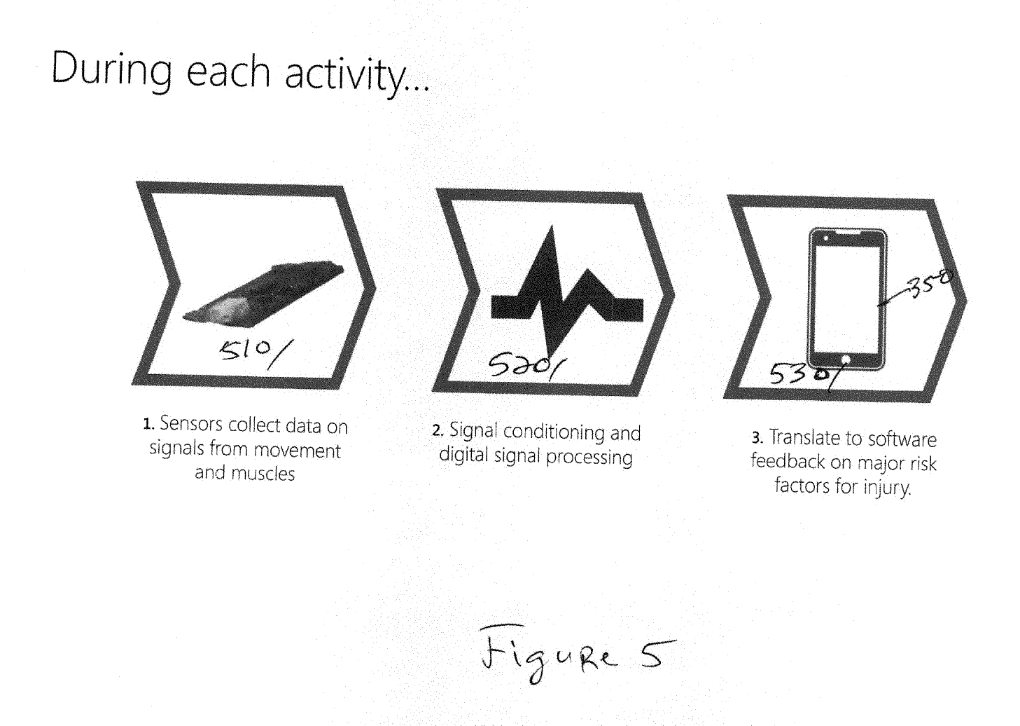

Referring now to FIG. 5, in certain aspects, embodiments enable the collection of sensor signals representing muscle activity and associated movement 510 for each activity to be monitored. Those signal are conditioned and processed and analyzed 520 for biological/physiological markers looking for potential injury, but before any injury actually occurs; these markers include, but are not limited to: muscle fatigue, appendage height, torque about any appendage joint, and reconstructing the raw motion path of the appendage to analyze movement technique. That resulting data is then transmitted 530 to a mobile device 350 for use in evaluating risk for potential injury. The format of the processed information may be further processed to be suitable for viewing based on the type of portable device (cell phone, laptop, computer, etc.) being used for viewing.

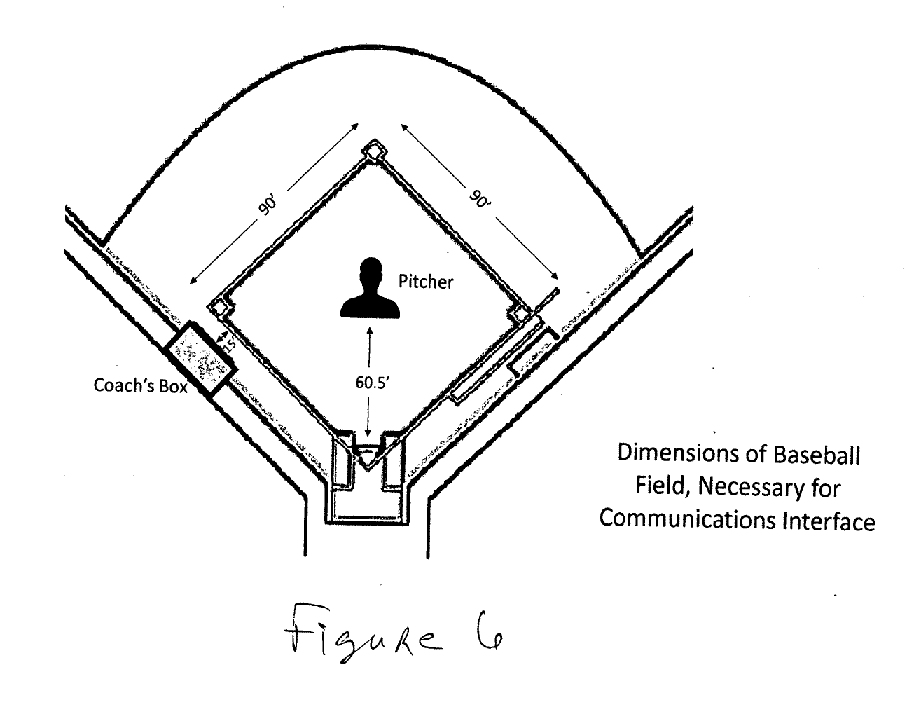

FIG. 6 depicts the dimensions of a baseball field and illustrates the representative distances over which a baseball embodiment useful for pitchers would be expected to transmit data for a sleeve 100 worn by a pitcher to a mobile device 350 associated with a pitching coach in a dugout.

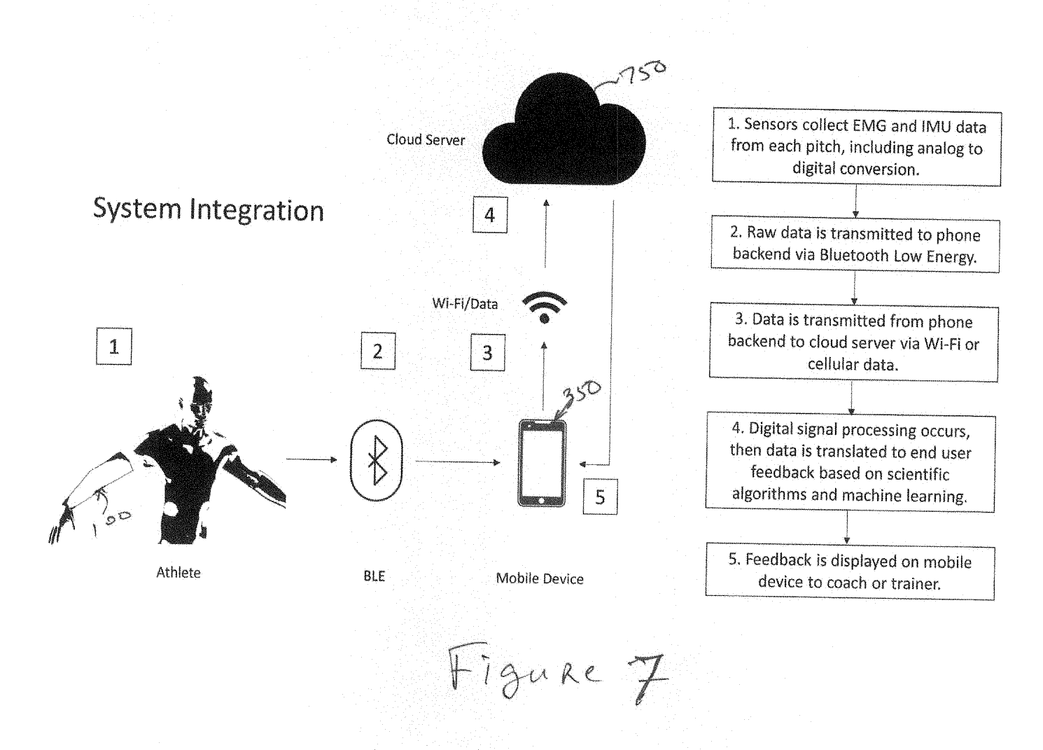

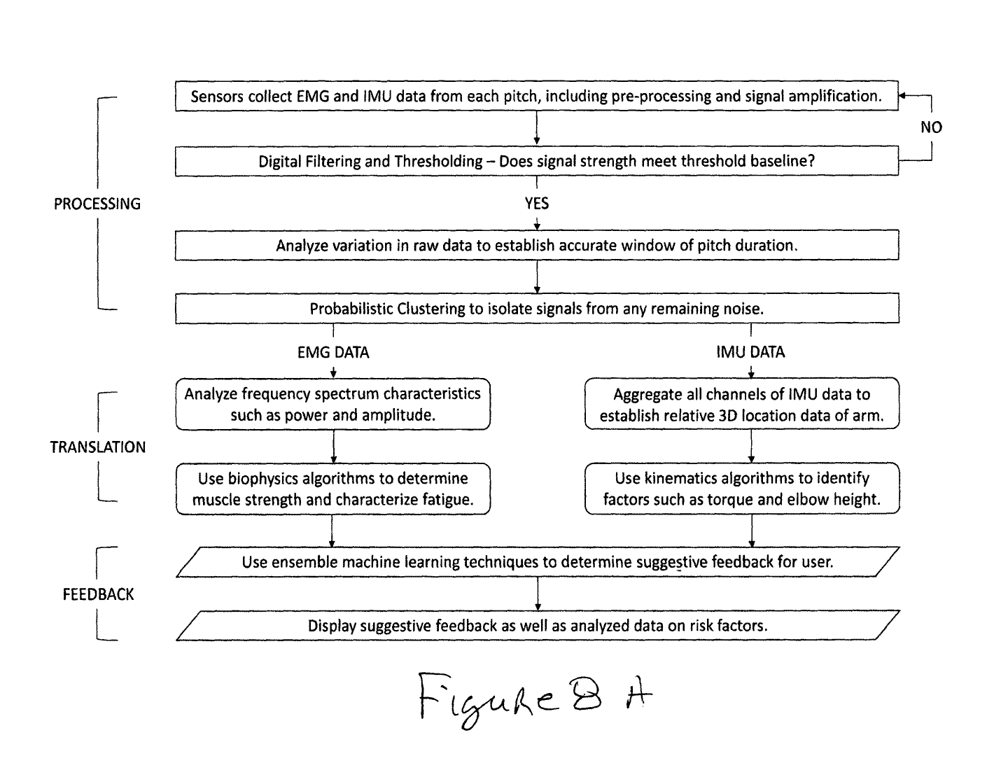

FIG. 7, depicts one method embodiment for a baseball application that collects sensor signals representing muscle activity and associated movement from sleeve 100. Those signals are conditioned and processed and the resulting data is then transmitted by the sleeve 100 to a mobile device 350, using low energy Bluetooth, or similar low energy methods for communication. The mobile device 350 transmits that data to a cloud server 750, where it is analyzed for biological/physiological markers looking for potential injury, but before any injury actually occurs, as well as looking for and providing feedback on pitching techniques. Again, these markers include, but are not limited to: muscle fatigue, appendage height, torque about the elbow joint, and reconstructing the raw motion path of the arm to analyze pitch movement technique. That resulting data and any associated feedback is then transmitted back to a mobile device 350 for use in evaluating risk for fatigue, potential injury and feedback on pitching techniques. Again, the analysis of the data for biological/physiological markers for potential injury, but before any injury actually occurs, as well as looking for and providing feedback on pitching techniques may optionally be performed by components in the sleeve 100, and the resulting information then transmitted to a mobile device 350 for use.

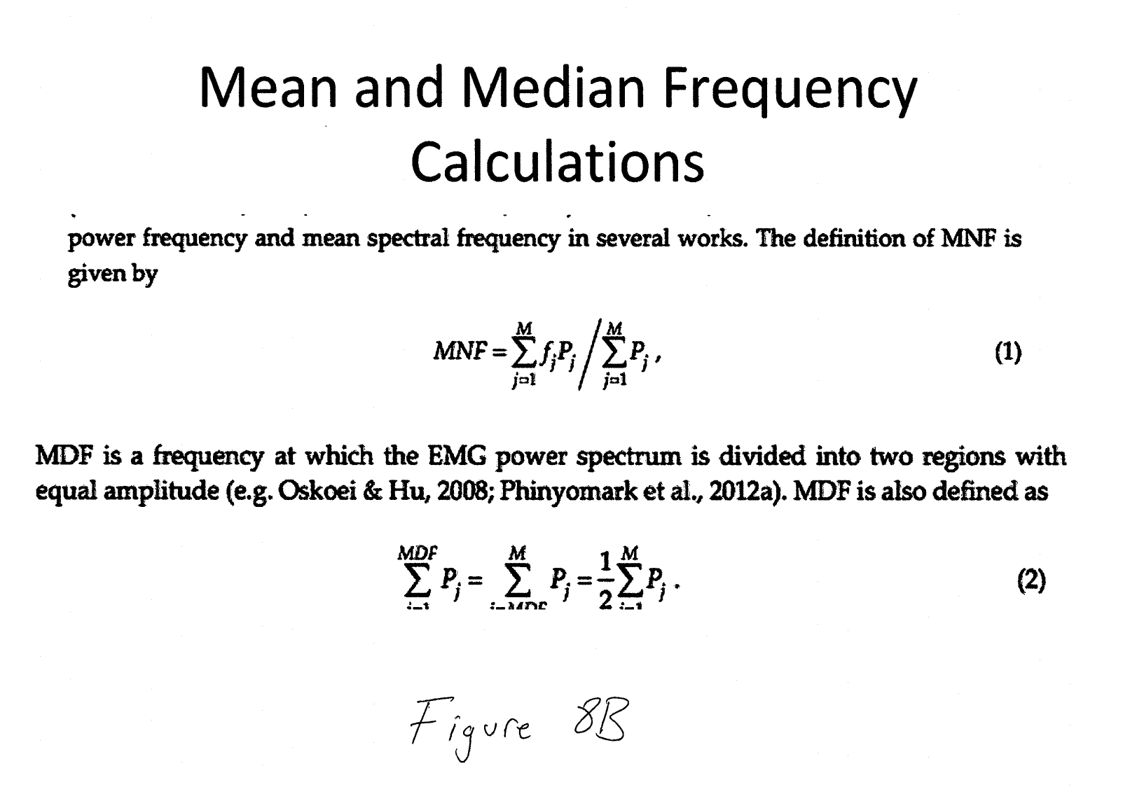

FIG. 8A depicts the computational aspects of one method embodiment in a system (preferably in software for ease of modification) which contains two main parts: digital signal processing to clean and process the raw data, and statistical/biological modeling to translate the data into useful end-user feedback. In the signal processing stage, the data channels will need to be filtered, classified, and isolated from motion artifacts and noise. To isolate the relevant data, an ensemble of digital signal processing and machine learning methods may be used. FIGS. 8B, C depict some representative intermediate calculations that may be used in FIG. 8A.

FIG. 8A for one embodiment initially collects EMG and IMU sensor data from each sensor, for each baseball pitch, that has been pre-processed and amplified, if necessary. The signals are then digitally filtered and a baseline threshold established to ensure the signal magnitude is sufficient for further processing, and if not, new signals are collected for a different time window. The signals are then analyzed for variations to establish a valid time window for the pitch. Then the signals are isolated using probabalistic clustering to remove any remaining noise signals. The data is then split into the EMG data or the IMU data for additional processing. The EMG data is analyzed in the frequency spectrum for characteristics, like for example, but not limited to, power and amplitude. Then biophysics algorithms are employed to determine characteristics, like for example, but not limited to, muscle strength and fatigue. The IMU data is separately aggregated to determine the relative positions of the arm during the pitch. Kinematics algorithms are then employed to determine factors, like for example, but not to torque and elbow height. The resulting data from the EMG and IMU analysis is then combined and processed using ensemble machine learning techniques, or other appropriate techniques, to determine feedback for the user. The feedback for correcting pitching motion errors and the processed and analyzed data, including any data concerning potential risks for injury, are then appropriately displayed for viewing by a user.

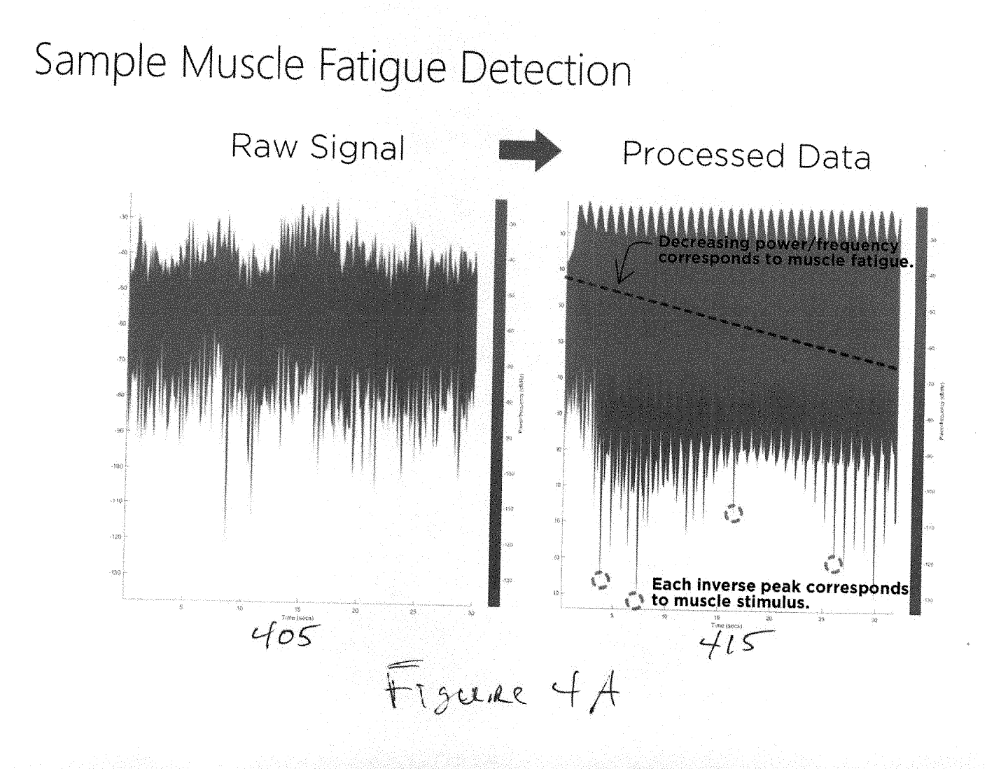

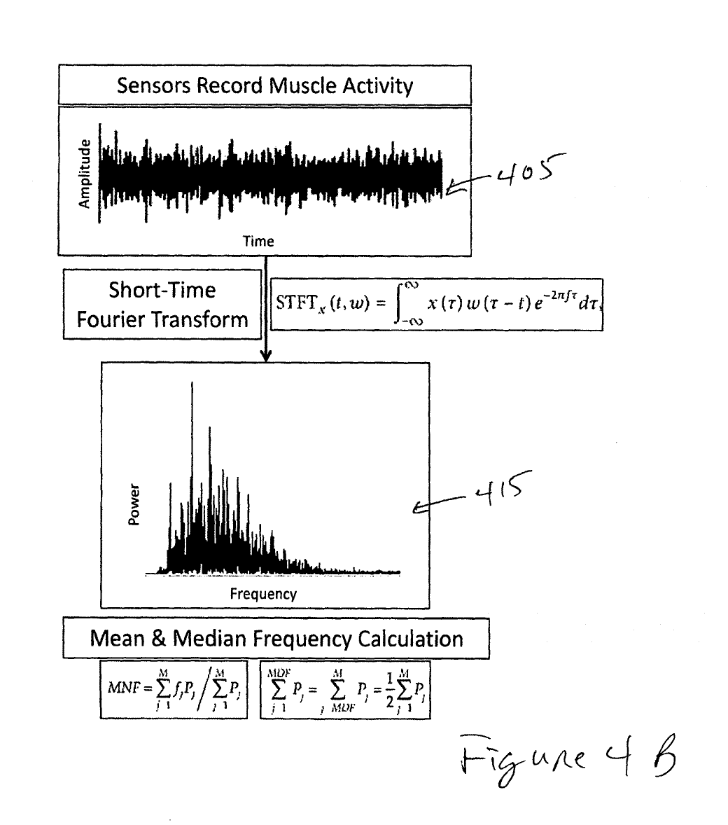

First, digital filtering in the frequency domain allows isolation of any actual nerve muscle activation signal from noise, then thresholding the filtered data allows baselines to be established that distinguish the actual time window of the pitching motion. FIGS. 4A, B depict raw muscle activity signals and their resulting frequency domain processed data from those signals, with FIG. 4A just showing the signals, and FIG. 4B also showing some representative intermediary processing steps.

This is augmented by use of the inertial measurement data. Multiple channels of IMU data are cleaned and filtered using extended Kalman filtering to get properly isolated 3-dimensional motion data. By analyzing the variation and progression in the relative location data of the motion sensors, and then comparing that against the raw muscle activation data, a more accurate window of physical activity can be established for proper isolation of data corresponding to the pitching motion. A probabilistic clustering-based machine learning technique such as Gaussian Mixture Modeling then is the final processing step for separating muscle activation signals from any remaining noise which may have initially passed for a significant signal in the filtering step.

Surface electromyography using non-gel based electrodes poses calibration challenges every time the device is worn. Sensor failures may be caused by external factors such as sweat and poor weather conditions. However, as described, digital signal processing can be augmented by ensemble machine learning methods such as convolutional neural networks. The use of inertial measurement data also provides secondary calibration to minimize the impact of such external factors. Also, there has been a rapid progression of materials research that can enable surface electrode material capable of a higher degree of accuracy than predecessor units, and is something which can be expected to continue to improve.

In the translation stage, that ‘clean’ data is converted into physiological feedback for each pitch, such as the reconstructed motion path, X/Y/Z relative location data, stress exerted on the arm, and monitoring muscle fatigue. By using data collected from multiple inertial measurement units, the relative positioning of the various sensors can be determined in order to establish the motion of the arm as a whole independent of environmental or other physiological factors. Using this method of relative positioning, kinematics algorithms can be used to analyze factors that have been proven as relevant in the understanding of fatigue and performance in physical activity, including, but not limited to: velocity of the arm, torque about the shoulder, elbow height relative to the shoulder, and a reconstruction of the throwing motion. Muscle fatigue is monitored by analyzing frequency spectrum characteristics of the processed signals. Specifically, the mean and median frequency of the EMG signal have been used extensively to determine muscle fatigue and estimate muscle force.

After repetitive or continual use, muscles begin to build up lactic acid, which results in a lower pH. This lowered pH correlates to a slowed conduction velocity, or slower muscle response. Taking the Fourier Transform of the muscle activity allows for a frequency domain analysis of the EMG signal. The slowed muscle response due to muscle fatigue results in a leftward shift in the frequency spectrum, or lower frequencies. The mean and median frequency techniques (MNF and MDF, respectively) have been heralded as the gold standard for capturing this leftward, lower frequency shift. Furthermore, time-dependent and min-max normalization techniques have been applied to the standard MNF and MDF techniques to better adjust for dynamic contractions and varied joint angles and forces. Therefore, applying these techniques to the acquired sEMG signal allows the characterization of muscle fatigue and the monitoring of the decrease in overall strength in the user's arm. It has been demonstrated that evaluation on the muscle-tendon activity during baseball pitching that muscles play a huge role in mitigating injury risk. Thus, the proper characterization of muscle fatigue is instrumental in providing feedback to the user in order to reduce the possibility of injury.

Muscle fatigue is generally a decrease in muscle tension/force and power production as the muscles tire. Muscles contract when the central nervous system (CNS) send signals to the individual motor units causing them to fire (action potentials). Motor unit action potentials (MUAPs) combine to cause muscle contractions; the more units recruited, the stronger the contraction. Muscle exercise (in this case throwing) causes lactic acid to build up, which results in a decrease in the conduction velocity (speed of CNS signal to muscles), leading to an increase in contraction times (and a decrease in firing frequency). Therefore, when measuring muscle activity—fatigue is shown by a decrease in the muscle activity frequency. This may be detected when the signals are converted to the frequency domain using Fourier transforms and a decrease in frequency is observed or a spectral shift to the left on an increasing frequency along the x axis plot.

The signal for muscle activity is gathered in real-time from the muscle sensors. Initial processing occurs to filter/smooth and condition the signal. Min-Max Normalization of the signal can also occur to reduce variation in the signal due to muscle geometry and joint angles. After the signal is conditioned in the time-domain, a short-time Fourier transform (STFT) produces the frequency response of the signal for power analysis. STFT is used to capture the signal in “sliding windows”, where segments of a certain window length are analyzed and then the window is shifted over (there is overlap) and repeated. For each power spectrum created through the STFT, the mean and median frequency (both or just one, may be determined) is calculated and subsequently analyzed. FIG. 8C illustrates how that calculation occurs.

The foregoing is currently the preferred method for performing this time and frequency-dependent fatigue analysis, but there are many techniques are available as is well known in the art. Different ways to condition the signal, normalize it, different forms of Fourier transforms and different forms of analyzing the frequency shifts (even different flavors of MNF and MDF). Use of these alternative techniques are considered to be part of the present disclosure.

After the translation stage, machine learning techniques then provide the final layer that allows each device to self-calibrate to its user, provide suggestive feedback on exertion limits and red flags, and allow for a greater depth of analysis. Using an ensemble of advanced regression methods and convolutional neural networks, each user's data set is analyzed to provide consistently evolving baselines of fatigue and harmful pitching motions. Gaussian Mixture Modeling is used across the larger repository of data to classify various types of pitches and pitching motions based on the feature set of IMU and EMG data. These classifications are then combined with the feature set of corresponding fatigue endpoint data to provide granular and stratified analysis including but not limited to: the fatigue impact of various types of pitches, performance data on pitch velocities, throwing motions relative to the optimum, and the suggested quantity of pitches remaining in a single throwing session.

Thus, a preferred embodiment provides a method for monitoring/analyzing biometric data from a user, by detecting EMG signals from at least one EMG sensor in contact with the skin of said user representing muscle activity for said user during at least one time window and said sensor is positioned on at least one preselected location on at least one appendage of said user using an elastic garment positioned on said appendage for containing said at least one EMG sensor in contact with said skin, amplifying, filtering and processing said detected signals to provide processed signals, converting said processed signals to digital format, processing said digital signals for conversion to frequency domain analysis, detecting IMU signals from at least one IMU sensor representing motion of said appendage associated with said muscle activity for said user during said at least one time window and said IMU sensor is positioned on at least one preselected location on said at least one appendage of said user using said elastic garment positioned on said appendage, processing said IMU signals for direction and orientation of said appendage, collecting said processed IMU signals and said digital signals, an optional transmitting step may be utilized here, processing and analyzing said collected signals for biological and physiological markers, and displaying at least a preselected set of said biological and physiological markers. The optional transmitting step includes transmitting said collected signals using a selected communications protocol for further processing and analysis, and then processing for markers, and appropriately displaying said markers.

The data transfer occurs via a wireless protocol such as bluetooth, as previously described earlier herein. This brings with it the challenge of data transfer with privacy and security. Several steps may be taken to mitigate any risks. Each user will have an access key associated with their software application executing on a mobile device. That access key can only be used on one device at any given time. The access key, aside from granting the user access to the mobile application, serves as the pairing password between the sensory sleeve and the mobile device. Once initially paired, all sleeve data that is transmitted becomes specific and accessible only to devices that are enabled under that access key.

To protect data privacy, the data which is transmitted from the sensory sleeve to the mobile device is client-side encrypted, which ensures that any data transmitted to the cloud server is encrypted and protected. In order to then process and analyze this data without needing to decrypt and expose the data to any malicious activity, a partially homomorphic encryption scheme enables cloud computation on the encrypted data. Homomorphic encryption allows for processing to be carried out on the encrypted data itself, thereby generating an encrypted result and ensuring that the data can only be decrypted on the client side, protecting it from third parties who aren't authorized to access the data.

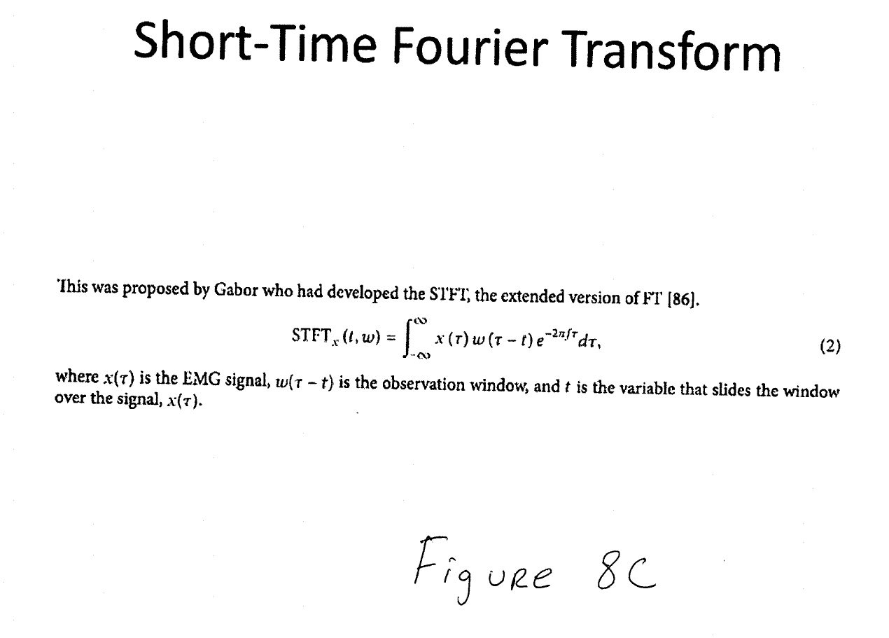

Referring now to FIGS. 9-14 simplified representative block diagrams of a system architecture and system components for one embodiment of the present disclosure are depicted. Referring now to FIG. 9, there may be seen a simplified representative block diagram of a system architecture for one embodiment of the present disclosure. More particularly, it may be seen that the system has a communication component 910 and associated antenna, a battery 920 and its supporting battery control component, and a plurality of sensor components 930 with their associated sensors. Preferably, each sensor and its associated component are one unit. The actual electrical devices or integrated circuits selected to be used for the individual components of this system are well known to those skilled in the art.

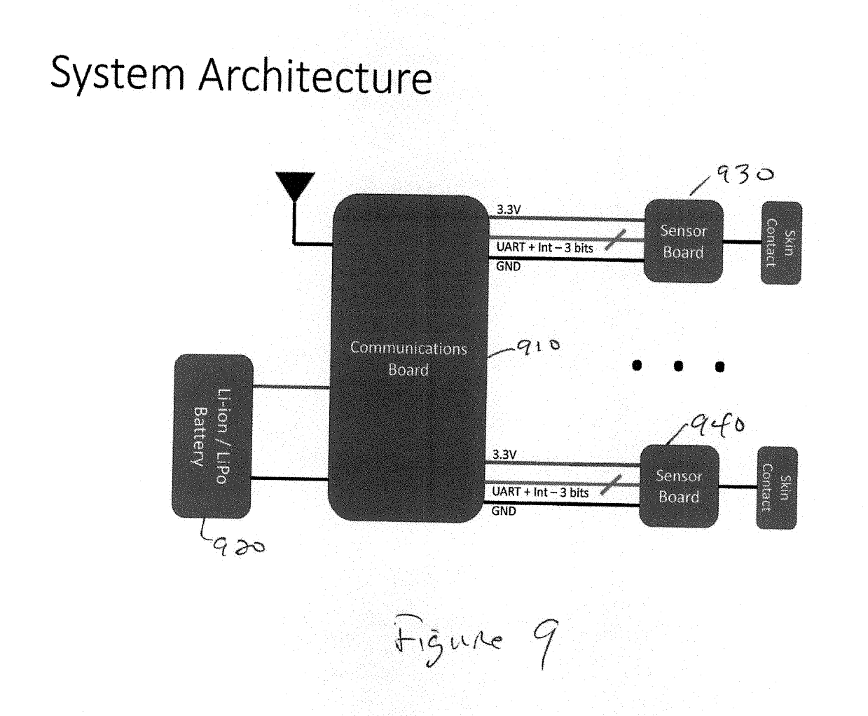

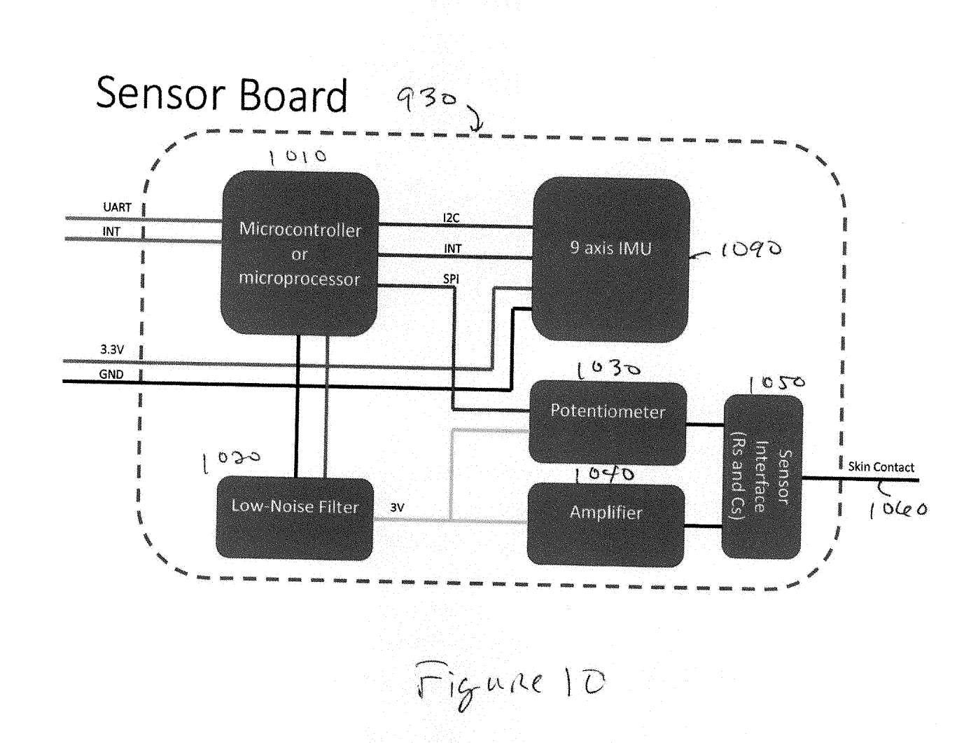

Referring now to FIG. 10 there may be seen a simplified representative block diagram of a combination sensor unit 930 for both EMG and IMU sensors for use in the system architecture depicted in FIG. 9. More particularly, it may be seen that the unit includes a microcontroller 1010 in addition to other circuit elements for separately providing proper voltages and amplification of output signals from the IMU sensor 1090 and the EMG sensor 1060. The EMG sensor 1060 is in contact with skin and its signals are very weak and easily corrupted by noise in any power lines for powering the sensor board (or sensor unit) 930. Accordingly the signal from an initial filtering interface 1050 is supplied to an amplifier 1040 for amplification. The amount of amplification is controlled by potentiometer 1030 which is in turn controlled by the microcontroller 1010. The amplified signal from the EMG sensor is then converted from analog to digital using an A/D converter (which may be a separate component or be part of the microcontroller) and the microcontroller then processes the signal as noted earlier herein.

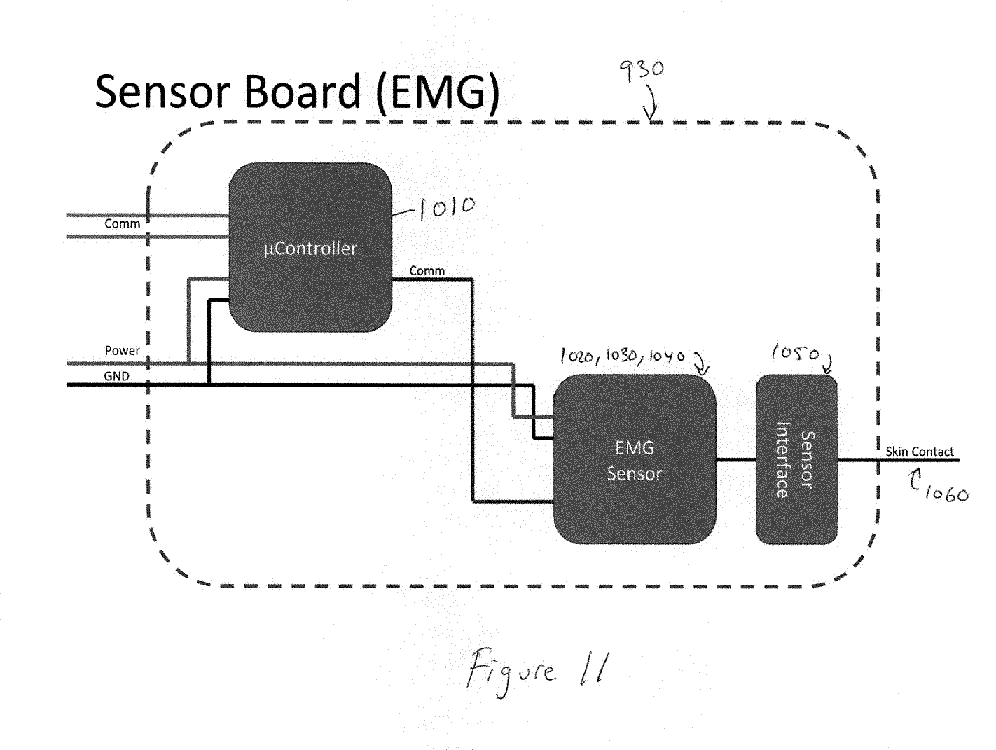

Referring now to FIG. 11 there may be seen a simplified representative block diagram of an EMG sensor unit for use in the system architecture depicted in FIG. 9. More particularly, it may be seen that the unit includes a microcontroller 1010 in addition to other circuit elements 1020, 1030, 1040, 1050 for providing proper voltages and amplification of output signals from the EMG sensor 1060.

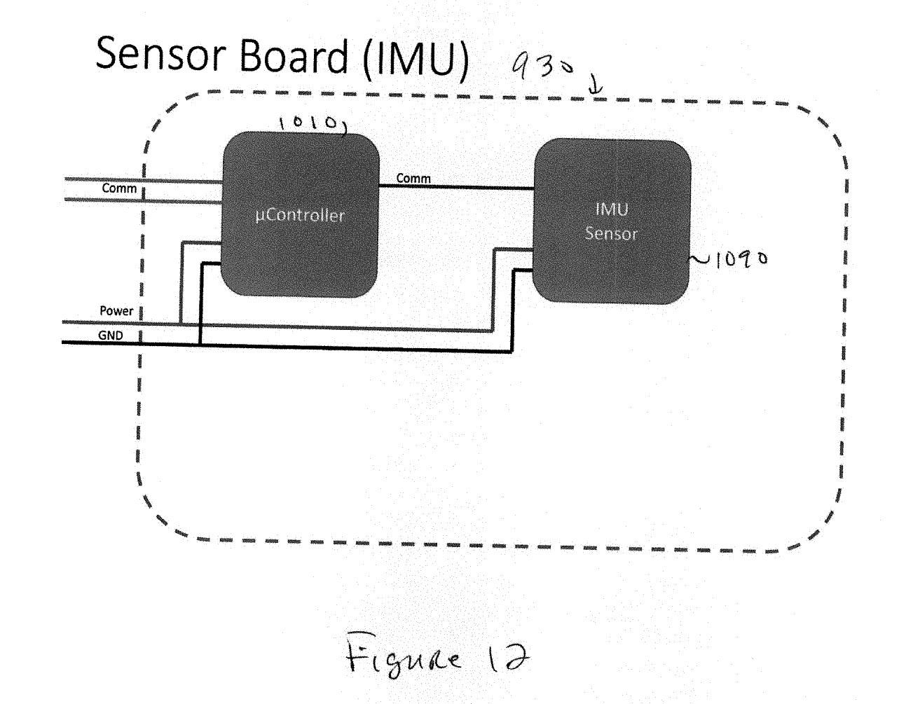

Referring now to FIG. 12 there may be seen a simplified representative block diagram of an IMU sensor unit for use in the system architecture depicted in FIG. 9. More particularly, it may be seen that the IMU sensor unit includes a microcontroller 1010 in addition to other circuit elements for providing proper voltages and amplification of output signals from the IMU sensor 1090.

Referring now to FIG. 13 there may be seen a simplified representative block diagram of a communications component for use in the system architecture depicted in FIG. 9. More particularly, it may be seen that the unit includes a multiplexor 1380 for receiving the output signals for the multiple sensor units 930 that may employed in the embodiments of the present disclosure. The multiplexor 1380 may in turn provide these signals to a microprocessor for additional processing, or to a component 1310 for transmitting these signals to a mobile device or optionally to a cloud server. In addition, a non-volatile memory component 1320 is shown for storing these signals. The circuitry for transmitting signals using wireless protocols or a wireless telephony system are well known to those skilled in the art. And a battery management component 1330 is depicted for controlling the battery 1340 for any charging and for supplying the appropriate voltages to the other components in the system.

The system of FIGS. 9-13 is an embodiment that is preferably initially calibrated for each user. This calibration includes selected appendage motions for calibration of the EMG signal amplifications and for resetting the clocks for all the microcontrollers to a common time base. The data form from each sensor unit includes the actual data (which may be pre-processed), a time window, the sensor type and the sensor location. The microcontroller in the communications component may be used to send a signal to the microcontrollers in each of the senor units to establish a common time base for the system.

In one aspect of the present disclosure a wearable physiologic sensor system is provided for a user, by providing a wearable elastic sleeve/garment worn by said user over at least one appendage for measuring/detecting appendage muscle activity, said sleeve, containing at least one EMG sensor in contact with the skin of said appendage and contained in an EMG sensor component located in said sleeve in a location over a muscle group associated with said appendage, at least one IMU sensor contained in an IMU sensor component located on said sleeve in a location on said appendage, wherein each EMG sensor component contains a microprocessor or microcontroller operatively connected to an adjustable amplifier, a filter for removing noise from the power supplied to said amplifier, an EMG sensor operatively connected to said amplifier, and an interconnection cable for receiving power and transmitting and receiving data, wherein each IMU sensor component contains a microprocessor or microcontroller, an IMU sensor, and an interconnection cable for receiving power and transmitting and receiving data, a communications component operatively connected to said interconnection cables and for transmitting data, containing a microprocessor or microcontroller operatively connected to transmitter circuitry, a non-volatile memory, a power management circuit and an input/output multiplexor, wherein said multiplexor is operatively connected to connectors for said interconnection cables and wherein said power management circuit is operatively connected to a rechargeable battery, and optionally an external processing component, containing a receiver for receiving said transmitted data and operatively connected to a microprocessor or microcontroller, wherein said microprocessor or microcontroller is operatively connected to a memory, a user interface and a display, and a power management circuit operatively connected to said receiver, microprocessor or microcontroller, memory, user interface and display and a power source.

### Other Embodiments

This sensory system can also be used in other applications beyond just a sleeve for baseball pitchers. Full-body units can be useful for high level athletes and in monitoring workouts in a general fitness setting, being able to analyze at the larger kinetic chain. Units for the leg around the knee, quads, and calves can help runners, football and soccer players, and be used towards any physical activity that places extensive repetitive strain on the legs. From a reactionary standpoint, this device can also help monitor and enable the consistent progression and execution of physical therapy regimen.

In one application, this system may be used to detect and monitor the risk of Anterior Cruciate Ligament (ACL) injuries in American Football. ACL tears are often non-contact injuries that occur on cutting movements, however there's ample scientific research now that shows that ACL injuries are not random occurrences. Muscle imbalance and fatigue surrounding the knee, improper movement mechanics, and overloading the joints are all connected factors which accumulate over time to lead to that one snapshot moment of injury. In fact, the most ACL tears in a single NFL season happened after the 2011 lockout, when there was shortened periods of preseason training, lending further credence to the role of proper biomechanical training in reducing the risk of such injuries. These are all physiological factors that can be monitored using a device in accordance with the teachings of this disclosure. The embodiment would comprise compression sleeves to be worn over the knees, underneath regularly worn pads. During the course of the activity, the coach or trainer would be able to monitor real time data on major risk factors for injury, such as the athlete's muscle fatigue, the stress exerted on their joints, and their running and jumping mechanics. The operation and analysis would be similar to that of the baseball application described earlier herein.

In another application, the system may be used to monitor the risk of arm injury for tennis players. Upper-limb injuries in tennis occur as a result of the high-velocity repetitive arm motions, typically owing to overuse and fatigue. Two such common injuries are tendonitis and lateral epicondylitis, otherwise known as “tennis elbow”. These injuries are the direct result of overuse and improper technique. As the rotational motion of the arm in tennis bears similarity to the usage of the arm in a baseball pitch, the sensory sleeve embodiment may be translated effectively to usage in tennis, enabling players and coaches to monitor a variety of factors to mitigate the risk of injury, including but not limited to: the athlete's serve technique, the force applied on each swing, shoulder rotation, and muscle fatigue.

In a therapeutic application, this system may be used in a reactive manner to aid the progression of physical therapy regimen. Currently, physical therapy is constrained by the limits of the technology used in hospitals, which tethers most of the monitoring ability to the local care site. However, with a device in accordance with the teachings of this disclosure, holistic clinical tracking system may be created, in which the user could work on physical therapy regimen from any location and be able to monitor their progress and activity as well as ensure that they do not re-aggravate any previous injuries. The primary caregiver would also be able to monitor the patient's progress, allowing them to stay updated, provide feedback, and track the patient remotely. The embodiment of such would likely comprise of various compression bands, sleeves, or straps to be worn over the training area of choice. This could also include a full body implementation as well.

While various embodiments of the present disclosure are described herein, it should be understood that they have been presented by way of example only, and not by way of limitation. Thus, the breadth and scope of the present disclosure should not be limited by any of the above described exemplary embodiments. Moreover, any combination of the above-described elements in all possible variations thereof is encompassed by the disclosure unless otherwise indicated herein or otherwise clearly contradicted by context. Those skilled in the art, having benefit of this disclosure, will appreciate that other embodiments may be devised that do not depart from the scope of the disclosure as claimed herein.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel. Accordingly, other embodiments, variations, and improvements not described herein are not excluded from the scope of the present disclosure.

What is claimed is:

1. A wearable biometric sensor for a user, comprising:
at least one EMG detector sensor, for detecting muscle activity in real-time,
at least one IMU detector sensor, for detecting motion in real-time,
an elastic wearable sleeve for positioning on an appendage of a user and for containing said EMG sensor in physical contact with said appendage and for containing said IMU sensor,
circuitry on said sleeve for detecting and processing signals from said EMG sensor,
circuitry on said sleeve for detecting and processing signals from said IMU sensor,
circuitry on said sleeve for collecting said processed signals, and
circuitry on said sleeve for processing and analyzing said collected signals for biological and physiologic markers.

2. The sensor of claim 1, further comprising:
circuitry on said sleeve for transmitting said collected signals to an external circuit including a processor for additional processing and analysis.

3. The sensor of claim 2, further comprising:
external display means for providing said analysis results for viewing.

4. The sensor of claim 2, further comprising:
processing said collected EMG signals in the frequency domain for detecting fatigue.

5. The sensor of claim 1, further comprising:
rechargeable batteries for powering said components and positioned in a different and separate location on said user from said sleeve.

6. The sensor of claim 1, further comprising:
non-volatile memory on sleeve or a separate location on user

7. The sensor of claim 1, further comprising:
processing said IMU data for appendage motions.

8. The sensor of claim 7, further comprising:
Integrating said appendage motions with said muscle activity to determine biologic and physiological markers.

9. The sensor of claim 8, wherein one or more of said markers is: muscle fatigue, joint height, torque about a joint, and raw appendage motion.

10. A method for training a user, comprising:
detecting muscle activity in a user appendage in real-time,
detecting motion of said user appendage in real-time,
processing said detected muscle activity to generate (useful) signals representing said detected muscle activity,
processing said detected appendage motion to generate signals representing said detected motion,
processing said signals to determine muscle fatigue in conjunction with said appendage motion, and
analyzing said signals to generate feedback on improving performance of said user without injury.

11. A method for monitoring/analyzing biometric data from a user, comprising:
detecting EMG signals from at least one EMG sensor in contact with the skin of said user representing muscle activity for said user during at least one time window and said sensor is positioned on at least one preselected location on at least one appendage of said user using an elastic garment positioned on said appendage for containing said at least one EMG sensor in contact with said skin,
amplifying, filtering and processing said detected signals to provide processed signals,
converting said processed signals to digital format,
processing said digital signals for conversion to frequency domain analysis,
detecting IMU signals from at least one IMU sensor representing motion of said appendage associated with said muscle activity for said user during said at least one time window and said IMU sensor is positioned on at least one preselected location on said at least one appendage of said user using said elastic garment positioned on said appendage,
processing said IMU signals for direction and orientation of said appendage,
collecting said processed IMU signals and said digital signals,
processing and analyzing said collected signals for biological and physiological markers, and displaying at least a preselected set of said biological and physiological markers.

12. The method of claim 8, further comprising,
transmitting said collected signals using a selected communications protocol,
and then processing said transmitted signals for markers.

13. A wearable physiologic sensor system for a user, comprising:
a wearable elastic sleeve/garment worn by said user over at least one appendage for measuring/detecting appendage muscle activity, said sleeve, comprising,
at least one EMG sensor in contact with the skin of said appendage and contained in an EMG sensor component located in said sleeve in a location over a muscle group associated with said appendage,
at least one IMU sensor contained in an IMU sensor component located on said sleeve in a location on said appendage,
wherein each EMG sensor component contains a microprocessor or microcontroller operatively connected to an adjustable amplifier, a filter for removing noise from the power supplied to said amplifier, an EMG sensor operatively connected to said amplifier, and an interconnection cable for receiving power and transmitting and receiving data,
wherein each IMU sensor component contains a microprocessor or microcontroller, an IMU sensor, and an interconnection cable for receiving power and transmitting and receiving data,
a communications component operatively connected to said interconnection cables and for transmitting data, comprising:
a microprocessor or microcontroller operatively connected to transmitter circuitry, a non-volatile memory, a power management circuit and an input/output multiplexor, wherein said multiplexor is operatively connected to connectors for said interconnection cables and wherein said power management circuit is operatively connected to a rechargeable battery, and an external processing component, comprising:
a receiver for receiving said transmitted data and operatively connected to a microprocessor or microcontroller, wherein said microprocessor or microcontroller is operatively connected to a memory, a user interface and a display, and a power management circuit operatively connected to said receiver, microprocessor or microcontroller, memory, user interface and display and a power source.
